(19) United States
(12) Patent Application Publication
(10) Pub. No.: US 2012/0264221 A1
(43) Pub. Date: Oct. 18, 2012

(54) METHOD FOR QUALIFYING A NON-PARTICULATE ADSORBENT BY MEANS OF A SECONDARY REACTION

(75) Inventors: Wolfgang Demmer, Goettingen (DE); Rene Faber, Goettingen (DE); Hans-Heinrich Hoerl, Bovenden (DE); Axel Thiefes, Hardegsen (DE)

(73) Assignee: SARTORIUS STEDIM BIOTECH GMBH, Goettingen (DE)

(21) Appl. No.: 13/515,679

(22) PCT Filed: Nov. 10, 2010
(86) PCT No.: PCT/EP10/06852

§ 371 Date: Jun. 13, 2012

(30) Foreign Application Priority Data
Jan. 8, 2010 (DE) 10 2010 004 190.4

(51) Int. Cl. G01N 21/78; G01N 33/20; G01N 21/79; G01N 33/00

(52) U.S. Cl. 436/79; 436/103

(57) ABSTRACT

The present invention relates to a method for the validation of a non-particulate adsorbent by secondary reaction and a kit for the validation of a non-particulate adsorbent by secondary reaction.

### BACKGROUND OF THE INVENTION

1. Field of the Invention

The present invention relates to a method for the validation of a non-particulate adsorbent by secondary reaction and a kit for the validation of a non-particulate adsorbent by secondary reaction.

2. Description of the Related Art

he present invention is based on the definitions described below. “Adsorptive substance separation” is understood to mean the separation of one or more components from a fluid phase by selective adsorption of this/these component(s) on a solid phase, the “adsorbent” (plural “adsorbents”). The field of the invention relates to substance separation in liquids, the liquid being called the “medium” below and the device in which the adsorption is performed the “adsorber”. Adsorbents are porous solids which via functional surface groups, which are called “ligands”, can selectively enter into bonds with certain components of fluids. As well as the long known “particulate” adsorbents, also called chromatography gels, other “non-particulate adsorbents” have become established, which are based on a matrix of an entirely different nature. These are so-called monolithic adsorbents consisting of a three-dimensional porous solid or support based on micro-porous membranes of various polymers. Two-dimensional adsorbents with the pores passing from one side to the other are described as adsorption membranes. According to the invention, target substance(s) and/or contaminant(s) are described as “adsorband” and used in the singular, although they can also consist of several different substances. The “capacity” of an adsorbent is understood to mean a quantitative measure for its uptake capacity for adsorband. The capacity is based on a defined quantity of adsorbent.

The present invention concerns non-particulate adsorbents. Some examples are mentioned below. In the state of the art, various non-particulate anion and cation exchangers are known. As examples, strong anion exchangers based on adsorption membranes such as Sartobind® Q from Sartorius Stedim Biotech GmbH, Mustang® Q from Pall Corp., Q Membrane from Natrix Separations or monoliths such as CIM® QA from BIA Separations are mentioned. Other examples are weak anion exchangers such as Sartobind® D from Sartorius Stedim Biotech GmbH, Chromasorb® from Millipore or CIM® EDA from BIA Separations. Other examples of negatively charged adsorption membranes are the strong cation exchanger Sartobind® S or weak cation exchanger Sartobind® C from Sartorius Stedim Biotech GmbH, the strong cation exchanger Mustang® S from Pall Corp., S Membrane from Natrix Separations or strong cation exchangers based on monoliths such as for example CIM® S03 or weak cation exchangers based on monoliths such as for example CIM® CM from BIA Separations.

The capacity of an ion exchanger is understood to mean a quantitative measure for its uptake capacity for exchangeable counter-ions. A distinction must be made between the total capacity and the usable capacity. While the total capacity states the total quantity of exchangeable counter-ions, the usable capacity relates only to that fraction which can be utilized under the particular operating conditions (e.g. pH of the solution, concentration of the solution, nature of the counter-ions). Adsorbands can be single molecules, associations or particles, which are preferably proteins or other substances of biological origin. Target substances can for example be recombinant proteins, such as for example monoclonal antibodies. Contaminants can for example be viruses, proteins, amino acids, nucleic acids, endotoxins, protein aggregates, ligands or parts thereof. The removal of contaminants the absence whereof is necessary or desirable for technical, regulatory or other reasons is described as “negative adsorption”.

Most contaminant removal applications are at present operated with conventional chromatography gels. These are particulate in form and are operated in the form of packings in columns. After filling of the column with the medium, a test for function and integrity follows. For this, the theoretical plate number/HETP and the asymmetry of the column packing are determined with suitable solutions of non-binding molecules such as acetone or cooking salt. On the basis of reference samples, the quality of the column packing and suitability for the chromatography step can be determined. The chromatography columns are markedly overdimensioned in order to achieve adequate flow rates. The columns are reused, which means a considerable cleaning and validation expenditure.

The implementation of chromatographic separations by means of adsorption membranes is also called membrane chromatography. The term adsorption membrane should be understood as a general term for various types of adsorption membranes, such as ion exchanger membranes, affinity membranes, hydrophobic membranes or activated membranes. Since filtration effects are most likely undesired, the pore sizes of the adsorptive membranes used on the industrial scale mostly lie in the range of >0.4 μm. In contrast to particulate adsorbents, adsorption membranes offer the possibility of forcing medium volume flow by application of a hydraulic pressure difference between the two sides of their surface, whereby instead of purely diffusive transport of the adsorband in the direction of a concentration gradient into the inside of the adsorbent, convective material transport is attained, which can take place very much faster with high volume flow rate. Thereby a disadvantage inherent to the particulate adsorbents, namely that with increasing adsorband particle size and increasing adsorband molecular mass the time necessary for establishment of the adsorption equilibrium increases considerably, can be avoided. Because of the described advantages of adsorption membranes, these are preferably used in process wherein the adsorband is present in the medium in very low concentration relative to the capacity of the matrix, so that a large volume of the medium can be processed per unit area of the adsorbent before exhaustion of its capacity.

Typical applications are in the field of negative adsorption, e.g. the removal of contaminants such as DNA, viruses, host cell proteins (HCP), CHOP (Chinese hamster ovary proteins) and endotoxins from antibody-containing solutions with positively charged adsorption membranes. This can (may) proceed irreversibly if the adsorbent is to be used only once. The breakthrough of contaminants is a critical factor in validated biopharmaceutical processes. The host cell proteins represent a broad spectrum of different cell proteins with different isolectric points (pI) and different size and affinity to the adsorbent. The concentration and composition of the contaminants depend on the expression system and on the upstream purification steps. Typical concentrations of host cell proteins in a protein A pool lie in the range 500-5000 ppm (ng/mg antibody) and in the range 50-500 ppm after a further CEX (cation exchange) step. The virus depletion is stated as the LRV (log reduction value). It corresponds to the negative base ten logarithm of the ratio of the virus concentration in the starting medium to the virus concentration in the filtrate. Hence an LRV of 5 means that 99.999% of the viruses have been removed by the adsorbent. Similarly, the depletion of endotoxins is stated as the LRV.

Adsorption membranes are in general used in modules/capsules which are also described as “membrane adsorbers”. They consist of a housing in which mostly one or preferably several layers of an adsorption membrane are installed. The adsorption membrane is sealed in the housing such that the flow is obligatorily through the membrane layers. The types resemble the modules customary in membrane filtration (e.g. wound module, stack module, etc.). The adsorber is as a rule supplied ready for connection, hence packing of the adsorber by the user is no longer necessary. The design and the shape of membrane adsorbers is adapted to the rapid mode of operation compared to the particulate chromatography columns. In the case of membrane adsorbers, the ratio of adsorption membrane stack height to incident flow area is orders of magnitude smaller than with chromatography columns. The quantities of adsorption membranes needed are as a rule markedly below those of chromatography gels. As a result, the influence of the dead volume and the adsorber periphery (tubes, pipes, connections, detectors) is also greater than with conventional chromatography columns. The validation methods used for chromatography, such as the determination of the plate number/HETP or the asymmetry of the column packing are thus rather insensitive and only usable for membrane adsorbers to a very limited extent.

The following criteria should be fulfilled and documented in the validation of an adsorbent installed in the process so that operation appropriate for the application is ensured and the regulatory requirements are fulfilled:
A. Are the correct functional groups present?
B. Is a sufficient quantity of functional groups present?
C. Is a sufficient quantity of functional groups attained during operation of the adsorbent?
D. Is the membrane structure, the membrane stack and the attachment of the membrane to the housing fault-free?

If all these four criteria are fulfilled for an adsorbent, according to the invention the integrity of this adsorbent is established.

Central to the validation of membrane adsorber systems by the manufacturer are measurements of different parameters, such as for example volume flow rate, binding capacity for model molecules, ligand density, mechanical stability, chemical compatibility and extractable substances. Analogously to the columns, the corresponding tests for functionality and integrity must also be conducted with the membrane adsorbers.

One of the methods used is an integrity test by means of a test device which was developed for sterile-filtering flat filters and filter candles. An example of a commercially available device is the Sartocheck® 4 from Sartorius Stedim Biotech GmbH. Here, the diffusion of air through a membrane stack wetted with water is determined and compared with an intact reference membrane stack. If the diffusion is above a predefined reference value, then a defect is present in the membrane stack. However, this method only yields information about the point D stated above and hence is only valid to a limited extent.

In one method (U.S. Pat. No. 7,281,410 B2, Oct. 16, 2007, Phillips, “Method for determining an effective Peclet number for a membrane device” and US Patent Application Publication US 2003/0089664 A1, May 15, 2003, Phillips, Membrane Adsorber Device), the determination of the Peclet number of a membrane adsorber is effected by the steps a) equilibration of the membrane adsorber with an equilibration buffer, b) loading of the membrane adsorber with a known concentration of a specific adsorband in an equilibration buffer, c) detection of the breakthrough of the adsorband as a function of time, loading volume and other suitable variables which are linked with the quantity of the adsorband loaded, d) analysis of the breakthrough curve in order to determine the relevant flow characteristics of the membrane adsorber by calculation of the sharpness of the breakthrough curve, and e) comparison of the results from step d) with a known intact membrane adsorber in order to determine the effective Peclet number. As the adsorband, for example tosylglutamic acid is used, the breakthrough whereof is detected by detection of the UV absorption.

A further method (US Patent Application Publication US 2008/0299672 A1, Dec. 4, 2008, Nochumson et al., “System and method for testing chromatography media and devices”) describes a method for the determination of the integrity of a chromatography membrane welded into a housing, wherein the membrane is subjected to pulsed application of an adsorband, e.g. adenosine monophosphate (AMP), under standard conditions, then the bound AMP is eluted with buffer solution and the concentration of the AMP in the eluate as a function of time is measured by UV absorption at 260 nm. The extinction coefficient-time curve thus obtained is compared with the extinction coefficient-time curve of an intact reference module. On occurrence of a defect (hole), in contrast to the intact reference module, early UV absorption occurs.

Both methods known in the state of the art use a “non-process” organic adsorband which is first adsorbed onto the adsorbent in a suitable buffer. In the method according to US 2008/0299672 A1, the adsorband must be eluted from the adsorbent. This represents a major and decisive disadvantage, since it must always be shown that the adsorband has been fully removed from the adsorbent and from the process medium or product. In some cases, the adsorband must be removed in a downstream process step. For regulatory, economic and process safety reasons, this represents a significant limitation. Further, the methods exhibit a relatively low sensitivity of detection via UV absorption and hence exhibit relatively low precision.

The present invention is based on the objective of providing a validation method for non-particulate adsorbents which enables highly sensitive, robust, simple, non-destructive testing of the integrity and functionality of non-particulate adsorbents. Preferably, aids (e.g. measuring instruments, test solutions) which mean no impairment of the function of the adsorbent or the product quality should be used for the validation.

### SUMMARY OF THE INVENTION

The invention describes a method which can detect faults or defects in non-particulate adsorbents by simple means, robustly, non-destructively and with very high sensitivity.

According to the present invention, the method for validation of a non-particulate adsorbent comprises the steps of: a) loading of the non-particulate adsorbent with an adsorband under conditions under which the adsorband is retained by the non-particulate adsorbent, b) detection of the adsorband that has broken through by secondary reaction wherein the negative base ten logarithm of the detected limit concentration is pD≧4, and c) comparison of the breakthrough characteristics with those of a non-particulate adsorbent of known integrity.

Advantageously, very small faults and defects can be detected with this method by secondary reaction by raising the negative base ten logarithm of the detected limit concentration to a value pD≧4. As a result, the retention capacity for contaminants, such as for example viruses, DNA and endotoxins can advantageously be tested with this method.

According to the present invention, any secondary reaction with which a relevant detection sensitivity for the adsorband that has broken through or parts thereof can be raised to the aforesaid detectable concentration is suitable. Examples of these are complex formation with a precipitation reaction or with a color reaction.

In the event of damage and/or a fault in the manufacture of a non-particulate adsorbent, the breakthrough and the characteristics thereof shift such that this occurs earlier, since the retention capacity of the non-particulate adsorbent is impaired and thus more adsorband can break through compared to with an intact non-particulate adsorbent. Surprisingly and advantageously, according to the present invention, owing to the sensitivity of the method, this can be detected even with only slight impairment of the adsorbent. This was not previously possible with UV absorption.

According to a preferred embodiment of the present invention, the loading with an adsorband is reversible. This ensures that after the validation the non-particulate adsorbent can be used for its relevant application without losses in performance.

Advantageously, in the process according to the invention, ions whose use does not impair the functionality of ion exchanger systems in most biotechnological applications can be used, and it is therefore possible to use the method as a so-called “pre-use” test before the actual use for example of an ion exchanger system. Accordingly, the adsorbent in the validation method according to the invention preferably comprises an ion exchanger system. According to the invention, the equilibration for the relevant process step follows directly after the validation of the adsorbent. The method according to the invention can advantageously be performed before (“pre-use”) and/or after (“post-use”) the use of the adsorbent. Furthermore, some process steps, such as for example disinfection of the adsorbent with sodium hydroxide solution, can be integrated into the validation method according to the invention, in that for example with an anion exchanger the first step is performed under disinfection conditions such as 1N NaOH for 30 mins. Similarly, the regeneration step after the use of the adsorbent, for example with 1N NaOH at elevated temperature, can be integrated into the method according to the invention.

In the method according to the invention, for example ions are applied onto an ion exchanger membrane adsorber under standard conditions until attainment of breakthrough of the ions. The breakthrough of the ions is detected with high precision and high sensitivity by use of a complexing agent and analytical detection of the ion complex.

The sensitivity of an analytical detection can be described by the limit concentration or pD value. The term limit concentration designates that concentration in g/ml of a substance to be detected at which the detection is still positive. More simply, instead of the limit concentration, the pD, which is defined as the negative base ten logarithm of the limit concentration, is introduced.

According to the present invention, the negative base ten logarithm of the detected limit concentration is pD≧4, preferably pD≧5, and more preferably pD≧6.

According to a preferred embodiment of the present invention, the ions as adsorbands comprise inorganic cations or inorganic anions. As inorganic cations in the method according to the invention, for example calcium ions from the group of the soluble calcium salts can be mentioned. An example of inorganic anions in the sense of the present invention are phosphate ions from the group of the oxyacids of phosphorus.

According to the present invention, it was surprisingly and advantageously found that on application of phosphate ions in the form of the free acid or water-soluble metal salts thereof onto an intact anion exchanger, the phosphate ions are adsorbed by the membrane before attainment of the break-through, and at the moment of the breakthrough of the phosphate ions the excess phosphate ions can be detected with great precision and high sensitivity as phosphorus molybdenum blue (C. H. Fiske and Y. P. Subbarow, J. Biol. Chem. 66, (1925), 375-400). The breakthrough of the phosphate ions is detected markedly sooner than with the known methods.

For potassium dihydrogen phosphate (KH₂PO₄) with the molecular mass of 136.09 g/mol, the phosphate detection as phosphorus molybdenum blue described in this invention has a limit concentration of 1 nmol/ml or 1.36×10⁻⁷ g/mol and corresponds to a pD of 6.9. Hence the sensitivity of this detection is higher than the known methods by several orders of magnitude.

It has been found that with the presence of a defect in the membrane adsorber, the breakthrough of the phosphate ions occurs early and markedly differs from the breakthrough of an intact reference adsorber module. Likewise, defects in multi-layer membrane adsorber units which are present only in one or some (e.g. three) layers are detected.

Analogously thereto, in the application of calcium ions in the form of the chloride onto a cation exchanger, it has been found that the calcium ions are adsorbed by the membrane before the breakthrough and the moment of the breakthrough of the calcium ions can be determined by detection of the excess calcium ions as poorly soluble calcium oxalate (G. Jander and E. Blasius, Introduction to Practical Inorganic Methods, 8^{th} Edn., S. Hirzel Verlag, Stuttgart 1968, p. 84) by measurement of the turbidity of the precipitating calcium oxalate.

For the calcium detection as calcium oxalate used in this method according to the invention, the pD is 6.5 and is thus far superior to the previously known methods.

The secondary reactions according to the present invention described above by way of example are a color reaction in the case of the phosphate ions and a precipitation reaction in the case of the calcium ions. According to the present invention the detection of these reactions is preferably effected by means of a photometric method. Here, according to the Lambert-Beer law, by means of preprepared solutions of defined dye or suspended matter concentration the extinction values at a wavelength are plotted against the corresponding concentration, a calibration line being thus obtained. Next, solutions of unknown concentration can be assayed and this determined on the basis of the calibration line.

On the basis of this very sensitive secondary reaction according to the present invention, it is possible to track the breakthrough of the adsorband with very high precision. Thus according to the invention it is possible to determine the integrity and functionality of non-particulate adsorbents exactly, even if the damage and/or impairment of the material to be tested is only slight, and an impairment linked therewith, for example in virus retention, is to be detected.

Finally, the present invention provides a kit for the validation of a non-particulate adsorbent, comprising an adsorband for loading of the non-particulate adsorbent under conditions under which the adsorband is retained by the non-particulate adsorbent, reagents for the detection of the breakthrough of the adsorband by secondary reaction and comparison data on the breakthrough characteristics of a non-particulate adsorbent of known integrity.

### BRIEF DESCRIPTION OF THE DRAWINGS

FIGS. 1 to 10 show calibration lines and breakthrough curves which are explained below in more detail in the context of the examples.

Here FIG. 1 shows a calibration line for the determination of the phosphate concentration as phosphorus molybdenum blue using the UV extinction at 820 nm.

FIG. 2 shows breakthrough curves on membrane adsorber Sartobind Q 100 for various phosphate solutions and the comparison of the breakthrough on the basis of the phosphate concentration and the pH in the outflow.

FIG. 3 shows breakthrough curves on 3-layer membrane adsorber stacks with artificially introduced faults for various hole sizes.

FIG. 4 shows breakthrough curves on 3-layer membrane adsorber stacks with artificially introduced faults, hole diameter 450 μm, wet and dry perforated.

FIG. 5 shows breakthrough curves on 3-layer membrane adsorber stacks with artificially introduced faults, hole diameter 450 μm, 1, 2 or 3 holes offset in the top (t), middle (m) and/or bottom (b) stack layer. Inflow side is at top.

FIG. 6 shows breakthrough curves on 3-layer membrane adsorber stacks with artificially introduced faults, hole diameter 450 μm, 1 single hole per stack, either in the top (t), middle (m) or bottom (b) layer. Inflow side is at top.

FIG. 7 shows a breakthrough curve on a 3-layer membrane adsorber stack on application of phosphate ions onto a membrane modified with polyallylamine.

FIG. 8 shows a calibration line for the photometric determination of the calcium ion concentration as calcium oxalate at 600 nm.

FIG. 9 shows a breakthrough curve on a 3-layer intact membrane adsorber stack with no hole, in particular the change in the calcium concentration with time on the basis of the turbidity in comparison to the conductivity C during the calcium ion application.

FIG. 10 shows a breakthrough curve on a 3-layer membrane adsorber stack with a hole (1100 μm), in particular the change in the calcium concentration with time on the basis of the turbidity in comparison to the conductivity C during the calcium ion application.

### DETAILED DESCRIPTION OF THE PREFERRED EMBODIMENTS

The present invention and further advantages deriving there-from are explained in more detail in the following description with reference to the embodiments described in the examples.

### EXAMPLES

### Examples for Anion Exchangers

For the studies on the anion exchangers, the following chemicals listed in Table 1 were used.

TABLE 1
Chemicals used for the studies on anion exchangers

| Substance | Manufacturer | Order No. | Lot |
|---|---|---|---|
| Ascorbic acid | VWR | 20150.231 | 07J030023 |
| Ammonium | ROTH | 3666.1 | 29782454 |
| heptamolybdate | | | |
| Sulfuric acid | Merck | 7312500 304 | K18980231 |
| NaH₂PO₄ | Merck | 106342.1000 | K91348242 732 |
| KH₂PO₄ | Merck | 104873.1000 | A672173 617 |
| NaOH | ROTH | 6771.1 | 27897776 |
| Na₃PO₄ | RIEDEL DE HAEN | 04277 | 72280 |

The water used was taken from a high purity water unit of the Arium® type from Sartorius Stedim Biotech GmbH.

### Example 1

### Determination of Calibration Line for Phosphate Determination as Phosphorus Molybdenum Blue

The following reagents were prepared:

Reagent A: 5 g of ascorbic acid were dissolved in 50 ml of water.

Reagent B: 6 N sulfuric acid (36 ml of 98% sulfuric acid were added to 180 ml of water).

Reagent C: 1.25 g of ammonium heptamolybdate were dissolved in 50 ml of water.

Reagent D: water.

50 ml each of reagent A, B and C were thoroughly mixed with 100 ml of reagent D. This working solution was freshly prepared before each series of determinations.

As standard solution, 0.68 g of KH₂PO₄ were completely dissolved in 1 liter of water; this corresponds to 5 mmol/l.

To obtain a concentration series, the standard solution was diluted to various concentrations in accordance with table 2, then 2 ml of working solution were added to 2 ml of each of these standard solutions and thoroughly mixed. The preparations were placed in a water bath at 70° C. for 10 mins. After this, the preparations were measured in a suitable glass cuvette in a spectrophotometer at 820 nm against a reagent blank (2 ml of water+2 ml of working solution).

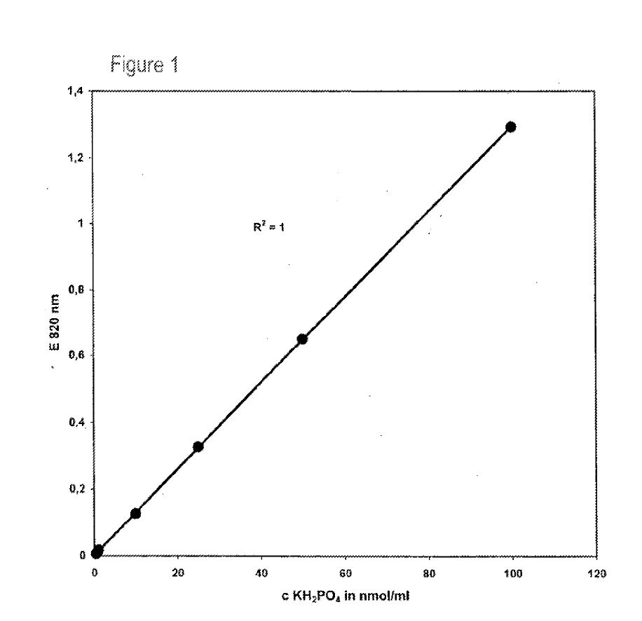

A typical standard curve is shown in Table 2 and FIG. 1. A linear dependence of the UV extinction on the quantity of phosphate used is seen. The coefficient of determination for the straight line R²=1.00.

TABLE 2
Values of calibration line for phosphate determination as phosphorus
molybdenum blue by UV extinction.

| Phosphate in | Extinction at |
|---|---|
| nmol/ml | 820 nm |
| 0 | 0 |
| 0.4 | 0.006 |
| 0.5 | 0.007 |
| 1 | 0.017 |
| 10 | 0.128 |
| 25 | 0.327 |
| 50 | 0.650 |
| 100 | 1.293 |

### Example 2

### Breakthrough Curve on a Membrane Adsorber Unit on Application of Phosphate Ions

A commercially available membrane adsorber unit with the name Sartobind® Q 100 containing 100 cm² of a strongly basic ion exchanger membrane with trimethylamine groups as ion-exchanging groups, from Sartorius Stedim Biotech GmbH, was attached with suitable adaptors to a chromatography system, type AKTA Prime plus from General Electric Healthcare. The system was operated according to the manufacturer's instructions.

The unit was deaerated according to the manufacturer's instructions and inserted into the system.

A program sequence for the chromatography system which contains the steps described below was written. The quantities used apply for the type described and are suitably adapted for other types and adsorber areas.

1. Rinsing of the adsorbent with 20 ml of a solution of 1 mol/l NaOH in water.

2. Washing of the adsorbent with high purity water until the conductivity in the outflow had fallen below 0.05 mS/cm. With the Sartobind® Q 100 type used, 50 ml of washing solution were programmed.

3. Application of a KH₂PO₄ solution (5 mmol/l) onto the adsorbent and simultaneous fractionation of the outflow into preferably 20 fractions of 2 ml volume.

4. Recording of the volume passed and the pH in the outflow by means of a flow cell for the pH.

The phosphate determination was performed on the fractions collected as described in example 1.

The measured values for the phosphate content in the outflow are represented graphically as a function of the filtrate volume in a breakthrough curve.

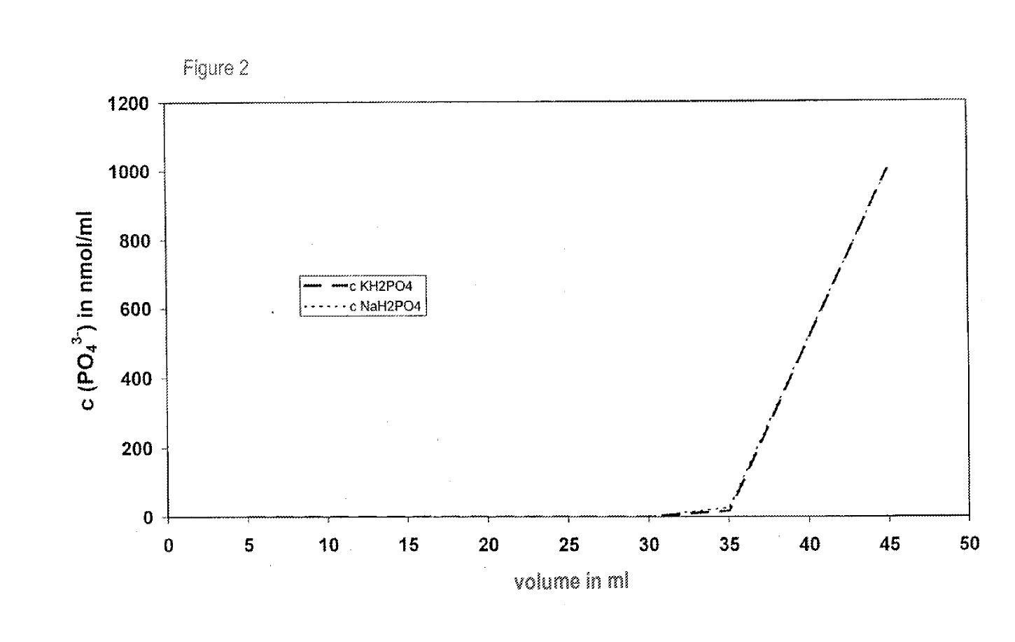

FIG. 2 shows the variation of the phosphate concentration with time in the outflow during the application of KH₂PO₄ solution or NaH₂PO₄ solution to the membrane adsorber in the inflow. At the start, all functional groups of the membrane adsorber are saturated by the prior rinsing with sodium hydroxide solution. On application of the potassium dihydrogen phosphate solution or sodium dihydrogen phosphate solution onto the membrane adsorber, the exchange of OH— ions for phosphate ions begins, during which OH— is released. When saturation of the membrane adsorber is attained, no further OH— ions are exchanged for phosphate ions and the excess phosphate ions pass through the membrane adsorber, i.e. they break through. If the variation of the concentration of the phosphate ions in the outflow with time is considered, then it is found that the phosphate ion concentration during the application to the membrane adsorber remains about zero, in order then to rise markedly on attainment of the saturation of the adsorber. The position of the phosphate breakthrough on the x axis can be detected very precisely on the basis of the phosphate ions appearing in the outflow. Further it is seen in FIG. 2 that both potassium and also sodium dihydrogen phosphate can be used. The results can be reproduced very precisely.

### Example 3

### Detection of Artificially Introduced Faults in Membrane Adsorbers by Plotting of the Breakthrough Curve for Phosphate Ions for Different Hole Sizes

A commercially available membrane of the Sartobind® Q type, a strong anion exchanger, order No. 94IEXQ42-001 from Sartorius Stedim Biotech GmbH was used. Three membrane disks with a diameter of 5 cm were punched out of the flat membrane sheet, laid into a 3-layer stack, placed in a clamping device in a suitable housing and integrated into the chromatography system as in Example 2. For the simulation of different defects in this membrane stack, before installation, holes with the diameters 450 μm, 600 μm and 1100 μm were punched both in dry membrane stacks and in membrane stacks wetted with water, using injection needles with flat-ground tips. Because of the flexible membrane matrix, the size and shape of the defects are not strictly defined.

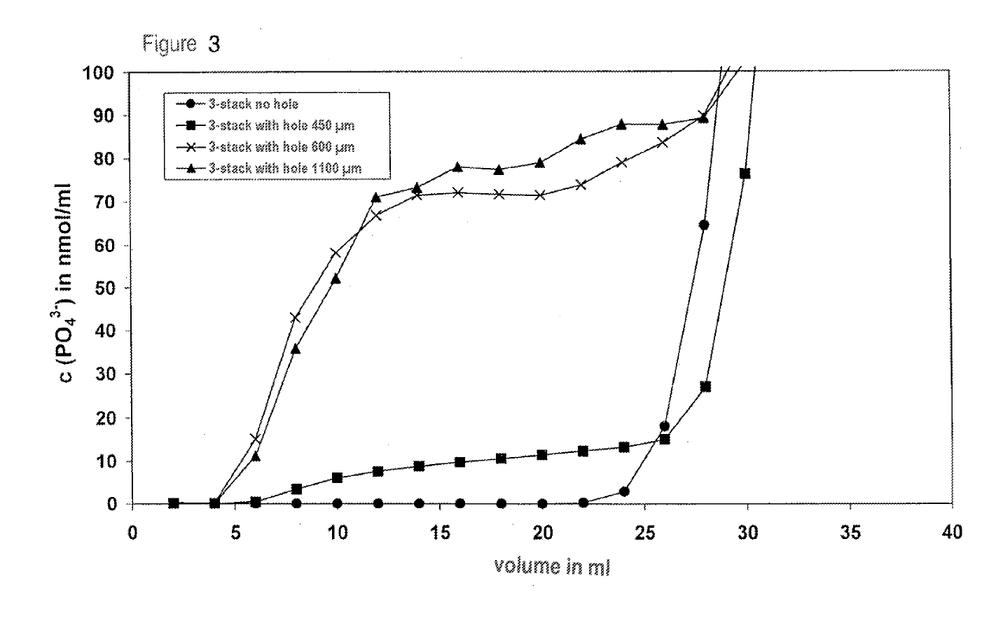

The application was effected with potassium hydrogen phosphate solution (1 mmol/l) and the outflow was fractionated into volumes of 2 ml each. The phosphate concentration was determined as in Example 1 and plotted as a function of the filtrate volume. A typical run is shown in Table 3 and FIG. 3.

TABLE 3
Breakthrough curves on 3-layer membrane adsorber stacks with artificially
introduced faults for different hole sizes

| | | | Hole | Hole | Hole |
|---|---|---|---|---|---|
| | | No hole | 450 μm | 1100 μm | 600 μm |
| | | C(PO₄³⁻) | C(PO₄³⁻) | C(PO₄³⁻) | C(PO₄³⁻) |
| Fraction | Vol ml | nmol/ml | nmol/ml | nmol/ml | nmol/ml |
| 1 | 2 | 0.00 | 0.08 | 0.23 | 0.00 |
| 2 | 4 | 0.00 | 0.00 | 0.15 | 0.23 |
| 3 | 6 | 0.00 | 0.45 | 11.21 | 15.15 |
| 4 | 8 | 0.00 | 3.41 | 35.76 | 42.95 |
| 5 | 10 | 0.00 | 6.06 | 52.12 | 58.11 |
| 6 | 12 | 0.00 | 7.58 | 70.98 | 66.74 |
| 7 | 14 | 0.00 | 8.79 | 73.18 | 71.36 |
| 8 | 16 | 0.00 | 9.77 | 77.88 | 71.97 |
| 9 | 18 | 0.00 | 10.61 | 77.27 | 71.59 |
| 10 | 20 | 0.00 | 11.52 | 78.86 | 71.36 |
| 11 | 22 | 0.23 | 12.42 | 84.39 | 73.79 |
| 12 | 24 | 2.88 | 13.26 | 87.80 | 78.94 |
| 13 | 26 | 18.11 | 15.08 | 87.73 | 83.56 |
| 14 | 28 | 64.55 | 26.97 | 89.24 | 89.70 |
| 15 | 30 | 147.35 | 76.29 | 102.05 | 107.27 |
| 16 | 32 | 196.97 | 170.45 | 143.71 | 156.06 |

FIG. 3 shows the breakthrough curves on 3-layer membrane adsorber stacks in which artificial defects (holes) had been introduced, compared to an intact stack. The markedly earlier breakthrough of the phosphate in the membrane stacks with a hole compared to the intact membrane stack with no hole can clearly be seen. Thus the significant rise in the concentration first begins with the stack with no hole at a volume of 24 ml, and already at volumes of 6-8 ml in the experiments with holed stacks.

A differentiation of the different hole diameters can also be discerned. Thus for the hole diameters of 1100 μm and 600 μm an immediate breakthrough is present after exit of the dead volume (ca. 5 ml). In the stack with a hole diameter of 450 μm the breakthrough occurs at somewhat higher volume and the curve is flatter.

The holes present can be unambiguously identified in this way in all the stacks.

### Example 4

### Detection of Artificially Introduced Faults in Membrane Adsorber Stacks by Plotting of the Breakthrough Curve for Phosphate Ions; Hole Diameter 450 μm, Perforated Wet and Dry

Membrane stacks were prepared and used as described in Example 3. Holes with a diameter of 450 μm were punched both in wet and also in dry stacks. Because of the flexibility and possible swelling effects in the wet state, the geometry of the resulting holes is not exactly defined.

TABLE 4
Breakthrough curves on 3-layer membrane adsorber stacks with
artificially introduced faults; hole diameter 450 μm, perforated wet
and dry

| | | | Hole 450 μm | Hole 450 μm |
|---|---|---|---|---|
| | | | wet | dry |
| | | No hole | perforated | perforated |
| | Vol | C(PO₄³⁻) | C(PO₄³⁻) | C(PO₄³⁻) |
| Fraction | ml | nmol/ml | nmol/ml | nmol/ml |
| 1 | 2 | 0.00 | 0.08 | 0.08 |
| 2 | 4 | 0.00 | 0.00 | 0.15 |
| 3 | 6 | 0.00 | 5.00 | 7.27 |
| 4 | 8 | 0.00 | 3.41 | 21.74 |
| 5 | 10 | 0.00 | 6.06 | 30.08 |
| 6 | 12 | 0.00 | 7.58 | 34.62 |
| 7 | 14 | 0.00 | 8.79 | 36.14 |
| 8 | 16 | 0.00 | 9.77 | 36.82 |
| 9 | 18 | 0.00 | 10.61 | 36.97 |
| 10 | 20 | 0.00 | 11.52 | 37.88 |
| 11 | 22 | 0.23 | 12.42 | 39.39 |
| 12 | 24 | 2.88 | 13.26 | 44.85 |
| 13 | 26 | 18.11 | 15.08 | 61.29 |
| 14 | 28 | 64.55 | 26.97 | 100.30 |
| 15 | 30 | 147.35 | 76.29 | 159.09 |
| 16 | 32 | 196.97 | 170.45 | 193.94 |

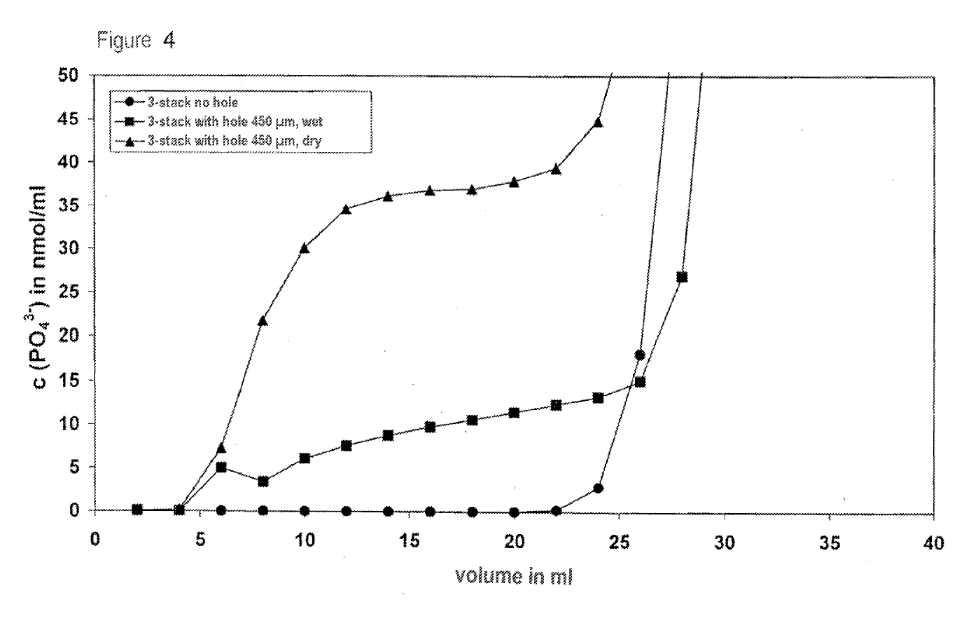

FIG. 4 shows the breakthrough curves on 3-layer membrane adsorber stacks in which artificial defects (holes) had been introduced, compared to an intact stack. The markedly earlier breakthrough of the phosphate in the membrane stacks with a hole compared to the intact membrane stack with no hole can clearly be seen. Thus the significant rise in the concentration first begins with the stack with no hole at a volume of 22 ml, and directly after exit of the dead volume of ca. 5 ml in the experiments with the defective stacks.

The markedly earlier breakthrough of the phosphate compared to the intact membrane stack with no hole can clearly be seen in the membrane stacks perforated in the wet and also in the dry state. The higher level of the breakthrough in the dry perforated stack is attributable to the greater shape stability of the hole in the dry membranes compared with the wet membranes.

The holes present can be unambiguously identified in this way in all the stacks.

### Example 5

### Detection of Artificially Introduced Faults in Membrane Adsorber Stacks by Plotting of the Breakthrough Curve for Phosphate Ions for Different Numbers of Faults; Hole Diameter 450 μm

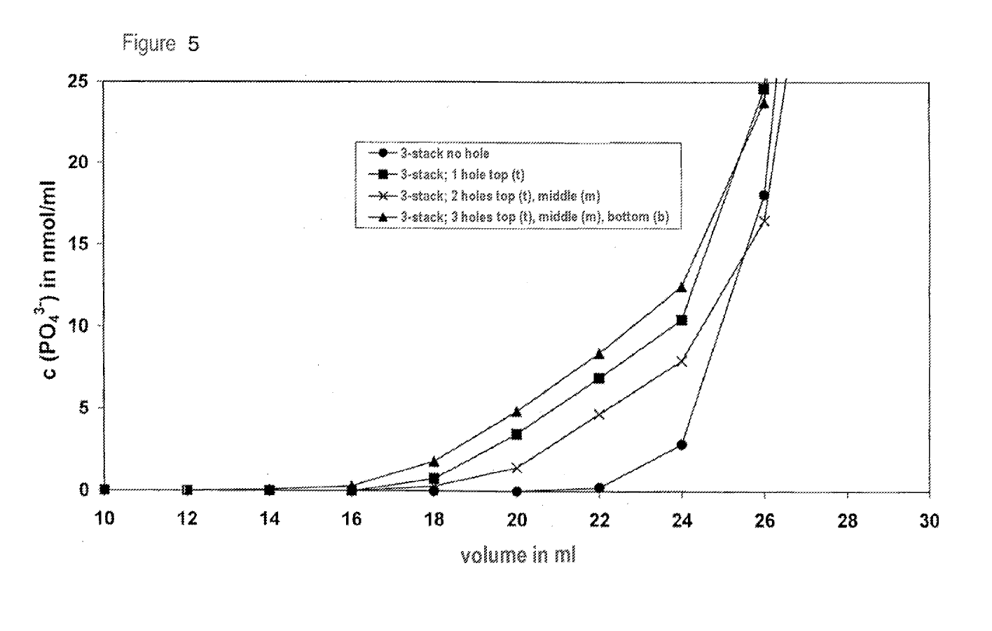

Membrane stacks were prepared and used as described in Example 3. Holes with a diameter of 450 μm were punched both in wet and also in dry stacks. The holes were pierced in accordance with Table 5 and FIG. 5 in the top (t), middle (m) or bottom (b) membrane layer. In each case the inflow side is the top side. The results of these experiments are shown in Table 5 and FIG. 5.

TABLE 5
Breakthrough curves on 3-layer membrane adsorber stacks with
artificially introduced faults; hole diameter 450 μm, 1, 2 or 3 holes
staggered in the stack top (t), middle (m) and/or bottom (b); inflow
side is top

| | | | | 2 holes | 3 holes |
|---|---|---|---|---|---|
| | | | 1 hole (t) | (t, m) | (t, m, b) |
| | | | dry | wet | wet |
| | | No hole | perforated | perforated | perforated |
| | | C(PO₄³⁻) | C(PO₄³⁻) | C(PO₄³⁻) | C(PO₄³⁻) |
| Fraction | Vol ml | nmol/ml | nmol/ml | nmol/ml | nmol/ml |
| 1 | 2 | 0.0 | 0.0 | 0.0 | 0.0 |
| 2 | 4 | 0.0 | 0.0 | 0.0 | 0.0 |
| 3 | 6 | 0.0 | 0.0 | 0.0 | 0.0 |
| 4 | 8 | 0.0 | 0.0 | 0.0 | 0.0 |
| 5 | 10 | 0.0 | 0.0 | 0.0 | 0.0 |
| 6 | 12 | 0.0 | 0.0 | 0.0 | 0.0 |
| 7 | 14 | 0.0 | 0.0 | 0.0 | 0.1 |
| 8 | 16 | 0.0 | 0.0 | 0.0 | 0.3 |
| 9 | 18 | 0.0 | 0.8 | 0.3 | 1.8 |
| 10 | 20 | 0.0 | 3.5 | 1.4 | 4.8 |
| 11 | 22 | 0.2 | 6.9 | 4.7 | 8.4 |
| 12 | 24 | 2.9 | 10.5 | 8.0 | 12.5 |
| 13 | 26 | 18.1 | 24.6 | 16.5 | 23.8 |
| 14 | 28 | 64.5 | 68.0 | 48.9 | 61.4 |
| 15 | 30 | 147.3 | 155.3 | 129.8 | 146.9 |
| 16 | 32 | 197.0 | 197.0 | 197.0 | 197.0 |

FIG. 5 shows the breakthrough curves on 3-layer membrane adsorber stacks with different number and position of the holes compared to an intact stack. Thus the breakthrough for the membrane stack with 1 hole lies at a volume of 18 ml, for the membrane stack with 2 holes at a volume of 20 ml and for the membrane stack with 3 holes at a volume of 18 ml, compared with the stack with no hole at a volume of 24 ml.

The holes present can be unambiguously identified in this way in all the stacks.

### Example 6

### Detection of Artificially Introduced Faults in Membrane Adsorber Stacks by Plotting of the Breakthrough Curve for Phosphate Ions for a Single Non-Piercing Hole per Stack

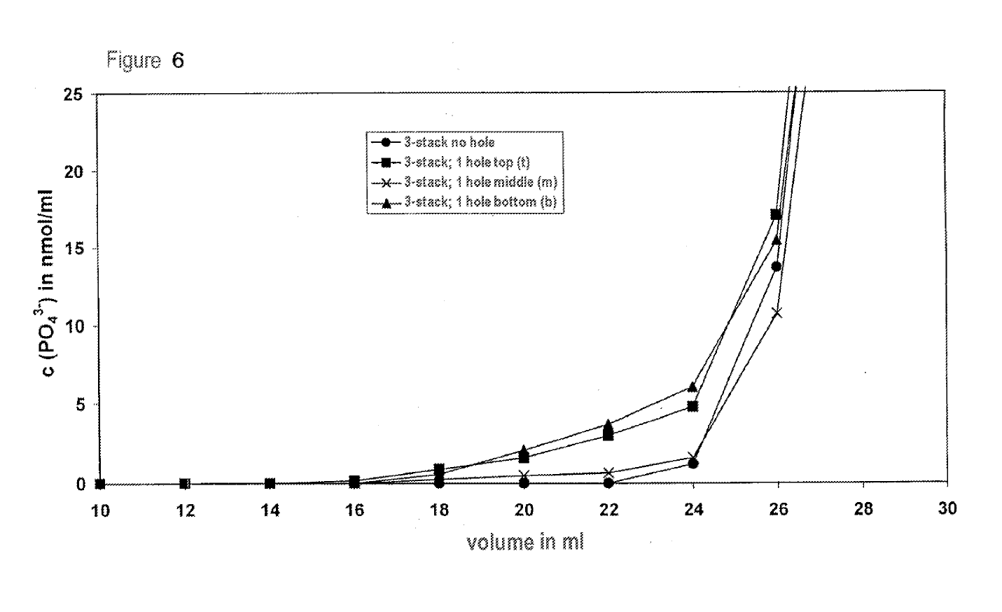

Membrane stacks were prepared and used as described in Example 3. A single hole (diameter 450 μm) was punched, as shown in Table 6 and FIG. 6, either in the top (t), middle (m) or bottom (b) membrane layer. The results of these experiments are shown in Table 6 and FIG. 6.

TABLE 6
Breakthrough curves on 3-layer membrane adsorber stacks each with a
single hole per stack, either in the top (t), middle (m) and/or bottom (b)
layer; inflow side is top; hole diameter 450 μm

| | | | 1 hole (t) | 1 hole (m) | 1 hole (b) |
|---|---|---|---|---|---|
| | | | dry | dry | dry |
| | | No hole | perforated | perforated | perforated |
| | | C(PO₄³⁻) | C(PO₄³⁻) | C(PO₄³⁻) | C(PO₄³⁻) |
| Fraction | Vol ml | nmol/ml | nmol/ml | nmol/ml | nmol/ml |
| 1 | 2 | 0.00 | 0.00 | 0.00 | 0.00 |
| 2 | 4 | 0.00 | 0.00 | 0.00 | 0.00 |
| 3 | 6 | 0.00 | 0.00 | 0.00 | 0.00 |
| 4 | 8 | 0.00 | 0.00 | 0.00 | 0.00 |
| 5 | 10 | 0.00 | 0.00 | 0.00 | 0.00 |
| 6 | 12 | 0.00 | 0.00 | 0.00 | 0.00 |
| 7 | 14 | 0.00 | 0.00 | 0.00 | 0.00 |
| 8 | 16 | 0.00 | 0.16 | 0.00 | 0.00 |
| 9 | 18 | 0.00 | 0.87 | 0.24 | 0.55 |
| 10 | 20 | 0.00 | 1.57 | 0.47 | 2.05 |
| 11 | 22 | 0.00 | 2.99 | 0.63 | 3.70 |
| 12 | 24 | 1.18 | 4.80 | 1.57 | 6.06 |
| 13 | 26 | 13.70 | 17.09 | 10.71 | 15.43 |
| 14 | 28 | 60.16 | 65.91 | 51.73 | 56.46 |
| 15 | 30 | 149.13 | 160.63 | 151.97 | 161.42 |
| 16 | 32 | 203.94 | 204.72 | 204.72 | 204.72 |

FIG. 6 shows the breakthrough curves on 3-layer membrane adsorber stacks with one single hole per stack, which is located in different layers, compared to an intact stack.

The considerably earlier breakthrough of the phosphate compared to the intact membrane can clearly be seen for all membrane stacks with a hole. Thus the breakthrough for the membrane stack with one hole in the top layer lies at a volume of 18 ml, for the membrane stack with one hole in the middle layer at a volume of 22 ml and for the membrane stack with one hole in the bottom layer at a volume of 20 ml, compared with the stack with no holes at a volume of 24 ml. Here the breakthrough with one hole in the middle layer is the least marked. This is attributable to the compensating action of the other two membrane layers.

The holes present can be unambiguously identified in this way in all the stacks.

### Example 7

### Breakthrough Curve on a 3-Layer Membrane Adsorber Stack with a Membrane Functionalized with Polyallylamine on Application of Phosphate Ions

A membrane from Sartorius Stedim Biotech GmbH, modified with polyallylamine, prepared as described in WO2009/127285 A1, example 21, was used. Three membrane disks with a diameter of 5 cm were stamped out of a flat membrane sheet, laid in a 3-layer stack, placed in a clamping device in a suitable housing and integrated into the chromatography system as in Example 2.

A program sequence for the chromatography system was written which contains the steps described below. The quantities used apply for the type described and are suitably adapted for other types and adsorber areas.
1. Rinsing of the adsorbent with 10 ml of a solution of 50 mmol/l HCl in water.
2. Washing of the adsorbent with 60 ml of high purity water.
3. Application of an Na₃PO₄ solution (1 mmol/l) onto the adsorbent and simultaneous fractionation of the outflow into preferably 20 fractions of 2 ml volume.
4. Recording of the volume passed and the pH in the outflow by means of a flow cell for the pH.

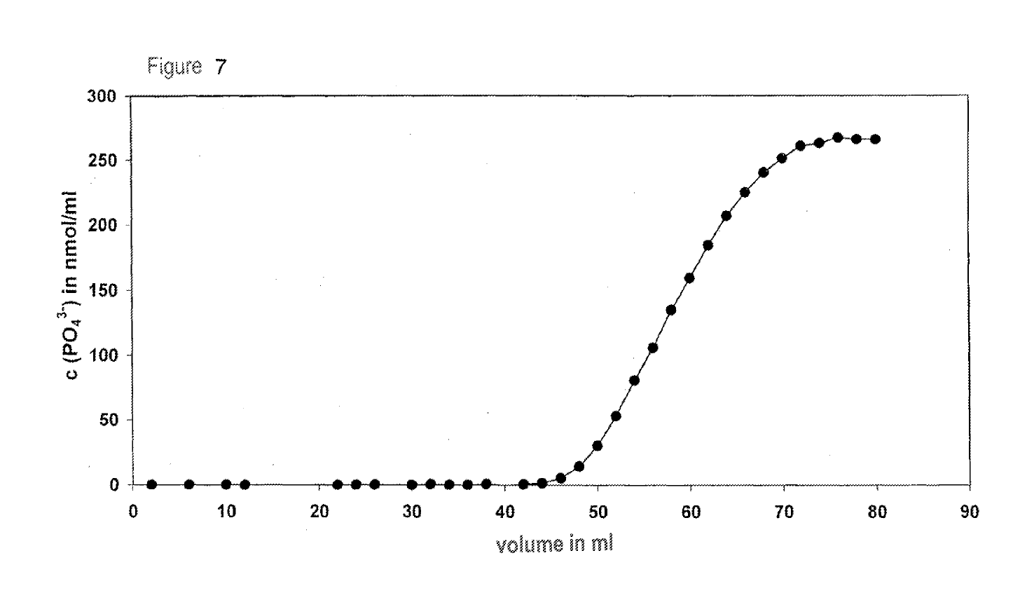

The phosphate concentration in the fractions collected was determined as described in example 1, and plotted as a function of the filtrate volume (FIG. 7).

FIG. 7 shows the variation of the phosphate concentration with time in the outflow during the application of Na₃PO₄ solution to the membrane adsorber in the inflow. At the start, the polyallylamine ligands of the membrane adsorber are present in the protonated form as NH₃⁺ groups with Cl⁻ ions as counter-ions due to prior rinsing with hydrochloric acid. On application of the sodium phosphate solution onto the membrane adsorber, the exchange of Cl⁻ ions for phosphate ions begins. When saturation of the membrane adsorber is attained, no further Cl⁻ ions are exchanged for phosphate ions and the excess phosphate ions pass through the membrane adsorber, i.e. they break through. If the variation of the concentration of the phosphate ions in the outflow with time is considered, then it is found that the phosphate ion concentration during application to the membrane adsorber remains about zero, in order then to rise markedly on attainment of the saturation of the adsorber. The position of the phosphate breakthrough on the x axis can be detected very precisely on the basis of the phosphate ions appearing in the outflow.

### Examples for Cation Exchangers

For the studies on the cation exchangers, the chemicals listed below were used.

| Substance | Manufacturer | Order No. | Lot |
|---|---|---|---|
| HCl | ROTH | P074.3 | 27896844 |
| CaCl₂ | MERCK | 102382.500 | TA1171782 250 |
| Ammonium oxalate | FLUKA | 09901 | 1343223 |
| | | | 22208210 |

The water used was taken from a high purity water unit of the Arium® type from Sartorius Stedim Biotech.

### Example 8

### Determination of Calibration Lines for the Calcium Determination as Calcium Oxalate

The following reagents were prepared:
Reagent E: 1 Mol/L HCl: 100 ml of 32% HCl were added to 900 ml of water
Reagent F: 2 mMol/L calcium chloride.
Reagent G: 1 mg/ml ammonium oxalate.
Reagent H: water.

2 ml of reagent G were added to 2 ml of test solution or appropriately diluted standard samples, and thoroughly mixed. The preparations were allowed to stand for 20 mins at ambient temperature. After this, the preparations were assayed in a suitable glass cuvette in a spectrophotometer at 600 nm against a reagent blank (2 ml water+2 ml reagent G).

Typical measured values for the standard curve are shown in

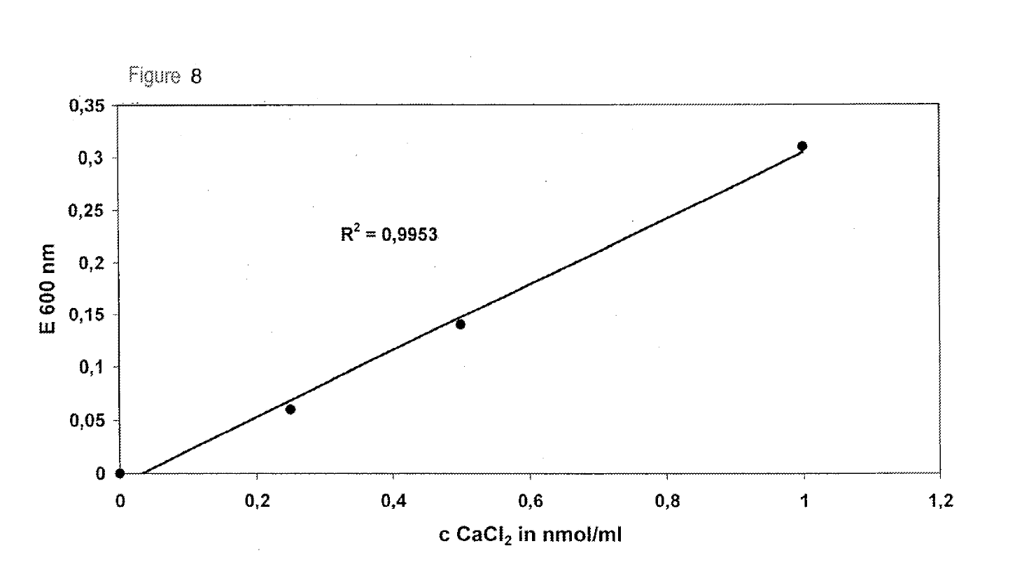

Table 8 and FIG. 8.

TABLE 8
Values of the calibration lines for the calcium determination as calcium
oxalate by photometric turbidity measurement at 600 nm

| μMol/L Ca²⁺ | E 600 |
|---|---|
| 0 | 0 |
| 0.25 | 0.06 |
| 0.5 | 0.14 |
| 1.0 | 0.31 |

Note:
mean value from 4 experiments.

### Example 9

### Breakthrough Curve on a Membrane Adsorber Stack on Application of Calcium Ions

A commercially available membrane of the Sartobind® S type, a strong cation exchanger, order No. 94IEXS42-001, from Sartorius Stedim Biotech GmbH, was used. 3 membrane disks with a diameter of 5 cm were stamped from the sheet, laid into a 3-layer membrane adsorber stack, introduced into a clamping device in a suitable housing and integrated into the chromatography system as described in Example 3.

A program sequence for the chromatography system was written which contains the steps described below. The quantities used apply for the type described and are suitably adapted for other types and adsorber areas.
1. Rinsing of the adsorbent with 20 ml of a solution of 1 mol HCl in water.
2. Washing of the adsorber/adsorbent with water until the conductivity in the outflow had fallen below 0.05 mS/cm. With the type used, 60 ml of washing solution were programmed.
3. Application of 40 ml of calcium chloride-containing solution onto the adsorber/adsorbent and simultaneous fractionation of the outflow into 20 fractions of 2 ml volume.
4. Recording of the volume passed, the conductivity and the pH in the outflow by means of suitable flow cells for conductivity and pH.

The determination of the calcium concentration was performed on the fractions collected as described in Example 7.

The measured values for pH, conductivity and calcium ion concentration in the outflow are represented graphically as a function of the filtrate volume in a breakthrough curve.

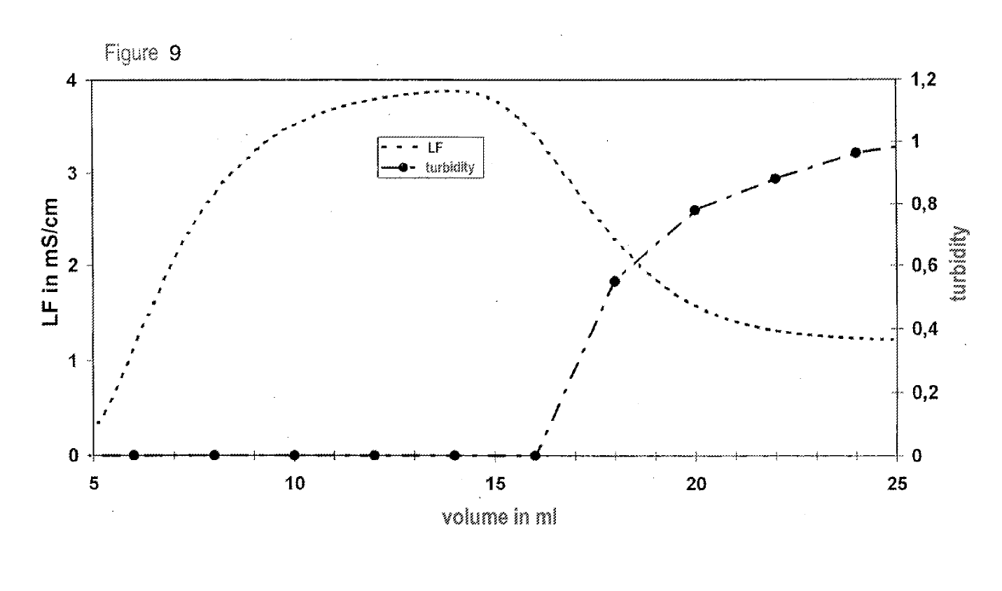

FIG. 9 shows the variation with time of the calcium concentration with time in the outflow during the application of calcium chloride solution to the membrane adsorber. At the start, all functional groups (sulfonic acid ligands) of the membrane adsorber are present in protonated form owing the prior rinsing with HCl. Through the rinsing with high purity water, the conductivity lies below 0.05 mS/cm. On application of calcium chloride solution onto the membrane adsorber, the exchange of the bound protons for calcium ions begins. The protons leave the adsorber with the counter-ions chloride as hydrochloric acid (HCl) and thereby the conductivity rises due to the increasing proton concentration in the outflow. If all protons have been replaced by calcium ions, i.e. the exchange capacity of the adsorber is exhausted, the breakthrough of excess calcium ions begins. The conductivity falls as the calcium ions exhibit a lower conductivity than the protons. The breakthrough is usually determined by the position of the inflection point of the conductivity curve.

If however the variation of the concentration of the calcium ions with time is considered, then it is found that the calcium ion concentration during its application to the membrane adsorber lies at about zero, in order then to rise markedly on attainment of saturation of the adsorber. It is seen that the breakthrough value which was determined by the calcium concentration in the outflow lies markedly before the breakthrough value which was determined by the conductivity. This means that the chemical determination of the calcium ion concentration is orders of magnitude more sensitive than the change in the conductivity.

### Example 10

### Detection of Artificially Introduced Faults in Membrane Adsorber Stacks by Plotting of the Breakthrough Curve for Calcium Ions

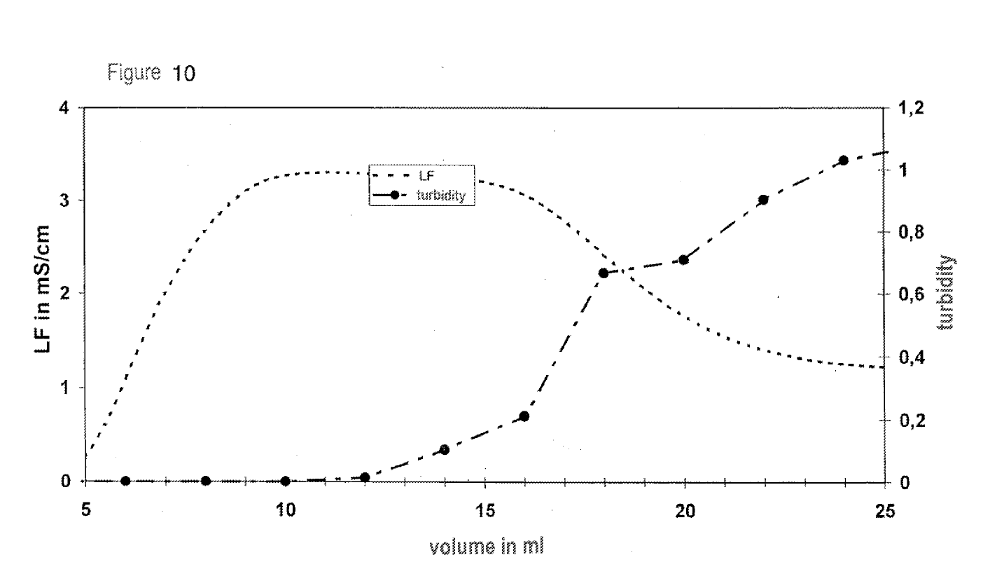

Membrane stacks were prepared as described in Example 9. For the simulation of a defect, through holes in different stacks were pierced before installation by means of an injection needle with a flat ground tip and the diameter of 1100 μm. During the application of the calcium chloride solution (2 mmol/l), the outflow was fractionated into 2 ml volumes. The calcium ion concentration was determined as in Example 9 and plotted against the fractionated volume. Furthermore, the change in the conductivity with time was plotted against the fractionated volume in FIG. 10.

Compared to the intact membrane stack in FIG. 9, it can clearly be seen in FIG. 10 that the breakthrough of the calcium ions, measured by the calcium concentration in the outflow, takes place markedly earlier, at ca. 12 ml, than with the intact membrane stack with no hole, at ca. 16 ml. The fault present can clearly be discerned in this manner.

If however the variation in the conductivity with time in FIG. 10 is considered, no changes can be discerned compared to the intact membrane stack in FIG. 9 with no hole. This means that through the conductivity measurement alone a fault of this order of magnitude, as represented by a hole with a diameter of 1100 μm, is not detectable under the experimental conditions given here.

1. A method for validation of a non-particulate adsorbent, comprising the steps of
loading the non-particulate adsorbent with an adsorband under conditions under which the adsorband is retained by the non-particulate adsorbent,
detection of the adsorband that has broken through by secondary reaction wherein the negative base ten logarithm of the limit concentration detected is pD≧4, and
comparison of the breakthrough characteristics with those of a non-particulate adsorbent of known integrity.

2. The method of claim 1, characterized in that the loading with an adsorband is reversible.

3. The method of claim 1, characterized in that the non-particulate adsorbent comprises an ion exchanger system.

4. The method of claim 3, characterized in that the adsorband comprises ions.

5. The method of claim 4, characterized in that the ions comprise inorganic cations or inorganic anions.

6. The method of claim 5, characterized in that the cations comprise calcium ions from the group of soluble calcium salts and the anions comprise phosphate ions from the group of the oxyacids of phosphorus.

7. The method of claim 1, characterized in that the secondary reaction comprises complex formation with a precipitation or color reaction.

8. The method of claim 7, characterized in that the precipitation reaction comprises the formation of a calcium oxalate complex and the color reaction comprises the formation of a phosphorus molybdenum blue complex.

9. The method of claim 8, characterized in that the detection of the precipitation and the color reaction is effected by a photometric method.

10. The method of claim 1, characterized in that the non-particulate adsorbent comprises a monolith or a polymer membrane.

11. The method of claim 10, characterized in that at least one ligand is bound to the polymer membrane.

12. A kit for validation of a non-particulate adsorbent, characterized in that the kit comprises an adsorband for the loading of the non-particulate adsorbent under conditions under which the adsorband is retained by the non-particulate adsorbent, reagents for the detection of the breakthrough of the adsorband by secondary reaction and comparison data of the breakthrough characteristics of a non-particulate adsorbent of known integrity.
